(19) United States
(12) Patent Application Publication
(10) Pub. No.: US 2005/0157887 A1
(43) Pub. Date: Jul. 21, 2005

(54) System for outputting acoustic signal from a stethoscope

(76) Inventor: Jong-Soo Kim, Seoul (KR)

Correspondence Address: CANTOR COLBURN, LLP, 55 GRIFFIN ROAD SOUTH, BLOOMFIELD, CT, 06002, US

(21) Appl. No.: 10/503,326

(22) PCT Filed: Nov. 15, 2002
(86) PCT No.: PCT/KR02/02136

(30) Foreign Application Priority Data
Jan. 31, 2002 (KR) 2002/5744

(51) Int. Cl. A61B 7/04; A61B 5/02

(52) U.S. Cl. 381/67; 600/528

(57) ABSTRACT

Disclosed is an output system for displaying an auscultatory sound signal such as heartbeat or respiratory sounds generated from the interior of a body in real time. The output system includes a stethoscope and a computer. The stethoscope includes at least a) a diaphragm to he brought into contact with a specific portion of a human body so as to vibrate according to a sound generated from the body, b) a microphone for converting the vibration of the diaphragm to an electrical sound signal, c) a signal process unit adjusting an auscultatory frequency band of the electrical sound signal from the microphone and outputting an auscultatory sound signal, d) a sound output unit for outputting a sound input from the signal process unit, and c) a signal output unit for transmitting the auscultatory sound signal output from the signal process unit to the outside. The computer displays an output signal received from the stethoscope, together with a standard sound pattern a user selects from standard sound patterns stored in a database.

### TECHNICAL FIELD

The present invention relates to a stethoscope, and more particularly to an output system for outputting a sound such as heartbeat or respiratory sounds generated from the interior of a body in real time.

### BACKGROUND ART

Generally, a stethoscope is used for collecting sounds, such as a heartbeat sound, from the internal organs of a human body to diagnose the health condition of a diagnosis subject. Such a stethoscope is composed of a bell for collecting sounds from the interior of a body, an earpiece for allowing a user to hear the collected sound therethrough, and a tube connected between the bell and the earpiece for transmitting the collected sound.

Such a stethoscope has a problem that, because it cannot collect a very faint sound, it cannot be applied to organs other than the heart, the lung, and several large-sized arteries or veins. The conventional stethoscope has other problems that only experts can handle it, and it only allows listening to a live sound from the body at the very moment of using it, so that it is difficult to accurately diagnose and treat the patient's condition due to the temporal and spatial limitations in sharing the diagnostic information.

In order to overcome such problems, there have been proposed a stethoscope equipped with a hearing aid or a minute-sound measurement meter for improving its diagnostic performance, and a stethoscope for allowing a number of students to hear simultaneously the diagnostic sound for medical educational purposes, and there has further been proposed an electronic stethoscope having a sound amplifying function for allowing the user to hear the diagnostic sound over the patient's clothes, i.e., without taking off the clothes.

Specifically, the electronic stethoscope is configured in such a manner that a sound generated from the body is collected and converted to an electrical signal, and then it is amplified to be output to a speaker, and the speaker converts the electrical signal to a corresponding diagnostic sound, enabling a user to hear the diagnostic sound such as heartbeat. Such an electronic stethoscope enables the collected sound to be transmitted to external equipment to also allow the patient to hear it, and it is recorded and then reproduced as needed. In addition, the electronic stethoscope allows patients to collect diagnostic sounds at their home and transfer them to a medical center or a specialist through the Internet or an ultra high-speed communication network, so as to receive various kinds of medical services.

However, this electronic stethoscope outputs only a sound, like the conventional stethoscope, so that it is likely that different doctors will provide different diagnoses based on the same sound, depending on their skills or subjective opinions. Accordingly, there is demanded a device allowing more objective diagnosis and monitoring of auscultatory sounds.

### DISCLOSURE OF THE INVENTION

Therefore, the present invention has been made in view of the above problems, and it is an object of the present invention to provide an output system for displaying an auscultatory sound signal, wherein the pattern of an auscultatory sound is output, in real time, through a monitor of a computer, simultaneously with the output of the auscultatory sound, thereby allowing more objective diagnosis and management of patients.

It is another object of the present invention to provide an output system for displaying an auscultatory sound signal, wherein an auscultatory sound pattern and a standard sound pattern according to the auscultatory status are simultaneously displayed to allow comparison therebetween, so that users can easily make a diagnosis of the auscultatory sound.

It is yet another object of the present invention to provide an output system for displaying an auscultatory sound signal, wherein auscultatory sound data is stored in a computer to be utilized as required, and an auscultatory sound signal is displayed for utilization in remote diagnosis using the Internet, etc.

In accordance with one aspect of the present invention, the above and other objects can be accomplished by the provision of an output system for displaying an auscultatory sound signal, wherein a collected auscultatory sound signal is transmitted to a computer for outputting a waveform representing the transmitted auscultatory sound signal, and the computer includes a memory unit for storing a database of standard sound patterns while being classified according to their categories, an information selection unit for selecting a standard sound pattern of the standard sound patterns stored in the memory unit, and a display unit for displaying the sound input pattern from the stethoscope, and the standard sound pattern selected by the information selection unit.

The output system according to said one aspect of the present invention has an advantage in that the user can analyze the health condition of a patient more definitely by referring to a standard sound pattern selected from the categorized standard sound patterns that are stored in the memory unit while being classified according to portions of the body of a patient.

In addition, the output system has an advantage in that, as well as the auscultatory sound obtained through the stethoscope is output to the outside through a speaker of the stethoscope, the auscultatory sound pattern and a standard sound pattern according to the diagnostic portions are separately displayed on one screen, so that, referring to the displayed patterns, the user can diagnose the health condition of patients more objectively and easily.

In accordance with another aspect of the present invention, the memory unit further includes an auscultatory sound database for storing at least auscultatory sound signals input from the stethoscope according to a manipulation signal by the user.

According to said another aspect of the present invention, the user can use the sound data stored in the memory as required. For example, the stored sound data can be useful in the case of comparing the present condition of a patient with the past condition to analyze the progress of the disease of a patient. In addition, a database storing auscultatory sounds according to diseases can be used as a presentation material for a conference or seminar.

On the other hand, according to said another aspect of the present invention, the patient can use the diagnostic result received from a doctor as evidence in cases where a medical accident occurs.

According to said one aspect of the present invention, the stethoscope for collecting vibrating sounds includes at least a) a diaphragm to be brought into contact with a specific portion of a human body so as to vibrate according to a sound generated from the body, b) a microphone for converting the vibration of the diaphragm to an electrical sound signal, c) a signal process unit for adjusting an auscultatory frequency band of the electrical sound signal is from the microphone and outputting an auscultatory sound signal, d) a sound output unit for outputting a sound input from the signal process unit, and e) a signal output unit for transmitting the auscultatory sound signal output from the signal process unit to the outside.

According to still another aspect of the present invention, the stethoscope further includes a recording-instruction portion for outputting a control signal allowing the computer to record the auscultatory sound signal input from a usual binaural-type stethoscope or the electronic stethoscope.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a view schematically showing the entire configuration of an output system according to a preferred embodiment of the present invention;

FIG. 2 is a view schematically showing the configuration of a stethoscope according to the preferred embodiment of the present invention;

FIG. 3 is a view schematically showing the configuration of a computer according to the preferred embodiment of the present invention;

FIGS. 4a and 4b are views showing the screens output through the output system according to the preferred embodiment of the present invention; and

FIG. 5 is a view schematically showing the configuration of a stethoscope according to another embodiment of the present invention.

### BEST MODE FOR CARRYING OUT THE INVENTION

Now, preferred embodiments according to the present invention will be described in detail referring to the drawings so that a person skilled in the art can easily carry out the present invention.

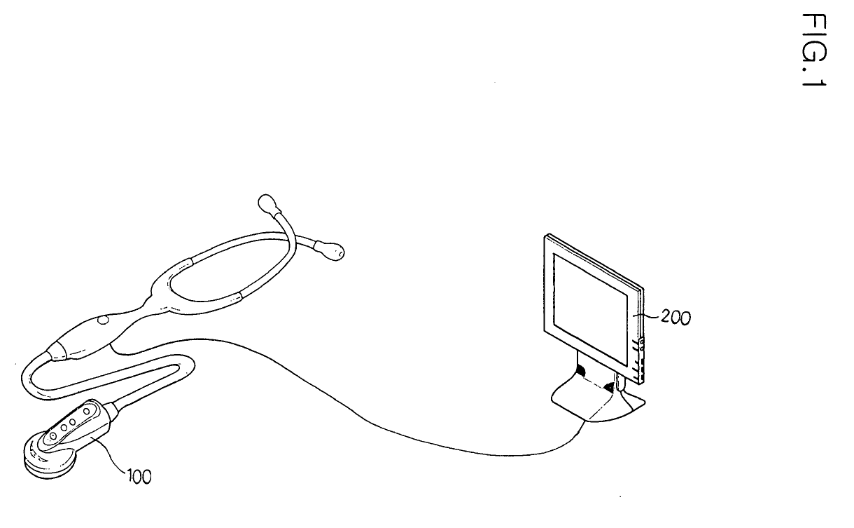

FIG. 1 is a view schematically showing the entire configuration of an output system for displaying an auscultatory sound signal according to the present invention. As shown in this figure, a stethoscope 100 includes a diaphragm vibrating according to a sound generated from a body, a microphone converting the vibration of the diaphragm to a corresponding electrical signal (referred to as “electrical sound signal”), a mode selection switch for selecting a sound signal, which a user wants to hear, from the electrical sound signal collected through the microphone, a volume switch for controlling the volume of the electrical sound signal, a speaker for outputting the electrical sound signal, an earpiece allowing a user to listen to the collected sound directly, and an output terminal providing a connection between the stethoscope 100 and a computer 200.

In such a manner, the stethoscope 100 according to a preferred embodiment of the present invention may be composed of an electronic stethoscope that converts the auscultatory sound signal to a digital signal, and transmits it to the computer 200. In addition, the stethoscope 100 according to another preferred embodiment of the present invention may be composed of a stethoscope commonly used in medical centers that transmits the auscultatory sound signal to the computer 200.

The computer 200, described below in detail, converts the sound signal from the stethoscope 100 to a signal having a corresponding waveform, and outputs the converted signal.

The applications of the present invention are not limited to diagnosis at restricted places such as a hospital and a public health center, but can provide a connection to portable computers such as a notebook or a web pad for outputting the auscultatory sound pattern, so as to achieve more accurate examination of the condition of a patient in an emergency vehicle or on a visit to the patient.

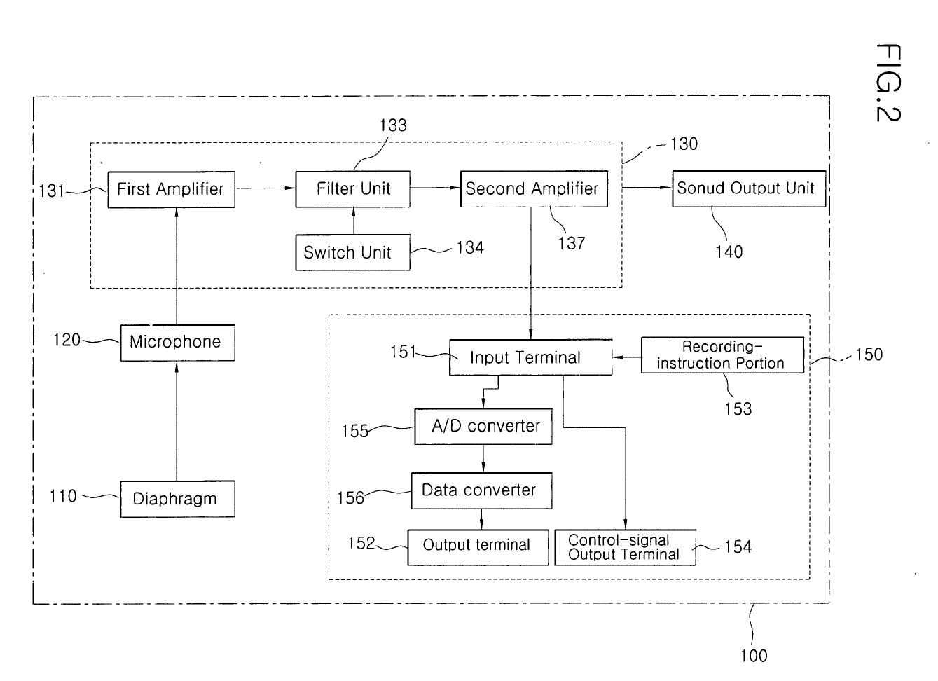

FIG. 2 is a view schematically showing the detailed configuration of the stethoscope according to a preferred embodiment of the present invention. As shown in FIG. 2, the stethoscope 100 includes a diaphragm 110 for collecting a sound such as a heartbeat sound, a microphone 120 for converting the collected sound to an electrical signal, a signal process unit 130 for processing the collected sound to produce an audible signal, a sound output unit 140 for outputting the sound, and a signal output unit 150 for transmitting the sound signal processed by the signal process unit 130 (hereinafter, referred to as “an auscultatory sound signal”) to a computer. In addition, the stethoscope includes a power supply unit (not shown).

The diaphragm 110 is composed of a vibrating plate and a sound collection portion. The vibrating plate is brought into contact with the skin of a human body for capturing a sound generated from the interior of the body. The sound collection portion functions to collect the sound. Thus, the diaphragm 110 functions to collect various sounds vibrating according to their sound pressure.

The microphone 120 converts a vibration detected by the diaphragm 110 to a corresponding electrical signal, and then outputs it to the signal process unit 130.

As mentioned above, the signal process unit 130 processes the electrical sound signal received from the microphone 120 to be audible to the users, and includes a first amplifier unit 131, a filter unit 133, a switch unit 134, and a second amplifier unit 137.

The first amplifier unit 131 amplifies a feeble electrical sound signal received from the microphone 120. Here, a sufficient degree of amplification is essential because the signal level of the electrical sound signal output from the microphone 120 is so low.

The filter unit 133 functions to extract a sound wave having a required specific band of frequencies from the signal output from the first amplifier unit 131, i.e., it separates a sound in the human-audible frequency range (20 Hz to 20 KHz) from the signal. Accordingly, sounds of ultra-low frequencies (not audible to humans) less than 20 Hz and sounds of high frequencies more than 20 KHz are filtered out to obtain the sounds of the frequency band actually audible to humans. Normally, heartbeat sounds are in the frequency range of about 20 Hz to 150 Hz, respiratory sounds are 200 Hz to 750 Hz, blood countercurrent frequencies of mitral valve or aorta are 170 to 900 Hz, and mitral stenosis frequencies are 25 Hz to 80 Hz.

The switch unit 134 enables the filter unit 133 to select only the signal in the specific band of frequencies. That is, based on operation signals from the switch unit 134, the filter unit 133 extracts a signal having the frequency range the user want to hear from the received electrical sound signal, and outputs the extracted signal to the second amplifier unit 137.

The second amplifier 137 amplifies the specific band of sound signal selected by the filter unit 133 according to the selection operation of the switch unit 134, so that the sound signal is converted to an audible sound signal having a capacity to drive the sound output unit 140.

As mentioned above, the signal process unit processes the sound signal to produce an audible sound signal via such operations of amplifying the electrical sound amplification, separating the signal according to frequencies, selecting a signal in a specific frequency range the user wants to hear, and re-amplifying the selected signal of specific frequencies. The specific auscultatory sound signal output from the signal process unit is output to the signal output unit for transfer to the computer.

On the other hand, the sound output unit 140 outputs a sound corresponding to the amplified auscultatory sound signal from the signal process unit 130, and includes a speaker, a sound collection portion, and an earpiece. The speaker outputs an auscultatory sound according to the auscultatory sound signal processed by the signal process unit 130. In addition, the auscultatory sound is output through the earpiece, and also output to the external so that a patient can hear his or her own auscultatory sound. The sound collection portion functions to collect the auscultatory sound output from the speaker so that the user can accurately hear the auscultatory sound. The collected auscultatory sound is transferred to the earpiece through a tube, so that the user can hear the auscultatory sound through the earpiece. That is, the tube serves as a guide for propagation the auscultatory sound output from the sound collection portion.

In such a manner, the stethoscope according to the present invention provides higher sound quality to the user, compared with the prior art stethoscope, because the speaker for outputting the auscultatory sound is provided on both split-portions of the body of the stethoscope, differently from the prior art stethoscope in which the speaker is provided inside the earpiece.

Meanwhile, the signal output unit 150 functions to output the auscultatory sound signal of analog type obtained through the stethoscope to external equipment so that the auscultatory sound signal can be used in medical equipment including a computer. According to one aspect of the present invention, the signal output unit 150 includes an input terminal 151 and an output terminal 151. According to another aspect of the present invention, the signal output unit 150 further includes a recording-instruction unit 153 and a control signal output terminal 154.

The input terminal 151 is connected to the second amplifier 137 for receiving the auscultatory sound signal output from the sound processor 130 to be output to the external equipment.

The output terminal 152 is connected to a sound card incorporating a microphone or line-in terminal of the computer so as to output the analog auscultatory sound signal input through the input terminal 151 to the computer.

In addition, according to further aspect of the present invention, during the diagnosis by the user, the recording-instruction unit 153 outputs a control signal to record the auscultatory sound signal without the user's manipulation of the computer.

The control signal output terminal 154 outputs the control signal input from the recording-instruction unit 153 to an input port (e.g., a game or serial port) of the computer.

The system according to such an aspect of the present invention has an advantage in that, during the user's examination, the auscultatory sound signal generated from the stethoscope can be transferred to the external equipment for checking the health condition of the patient at any desired time. In addition, the present invention facilitates its use in that, during the diagnosis, the auscultatory sound signal input to the computer can be recorded without the manipulation of the computer.

Meanwhile, it is preferable to install a program in the computer whereby the auscultatory sound signal input to the microphone or line-in terminal of the sound card in the computer is converted to a digital signal, according to a control signal from the stethoscope, and recorded in an auxiliary storage device such as a hard disc or transferred to a remote place through the Internet.

In addition, according to an additional aspect of the present invention, the signal output unit 150 further includes analog-to-digital (AD) converter 155 and a data converter 156. The AD converter 155 converts the analog auscultatory sound signal filtered by the signal process unit 130 to a digital data. That is, the AD converter converts the electrical signal of analog type to a digital data usable in digital electronic medical equipment.

The data converter 156 converts the auscultatory sound data digitalized by the AD converter 155 to a data transmittable to the digital electronic medical equipment such as a computer. That is, the data converter 156 produces a data having a signal level and a transmission format necessary for communication with the computer.

The data converted by the data converter 156 is transmitted to the computer through the output terminal 152. The output terminal 152 is composed of a communication port enabling data transfers to the computer. Currently used communication ports are, for example, RS232C, IEEE488, a print port, a USB port, or a wireless communication port using an infrared or RF signal.

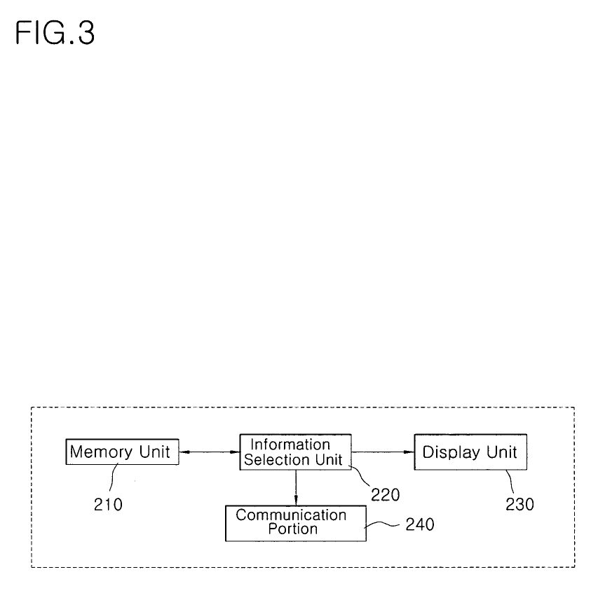

FIG. 3 is a view schematically showing the configuration of the computer according to a preferred embodiment of the present invention. As shown in this figure, the computer 200 includes a memory unit 210, an information selection unit 220, and a display unit 230. In another aspect of the present invention, the computer 200 further includes a communication portion 240. Although not shown, the computer includes an input unit for receiving the auscultatory sound signal output from the stethoscope.

The memory unit 210 includes a standard sound pattern database containing ideal sound patterns (hereinafter, referred to as “standard sound patterns”) of the internal organs of a body, while being classified according to the patient's age. The memory unit 210 further includes an auscultatory sound database that stores at least auscultatory sound signals input from the stethoscope according to a manipulation signal of the user. On the other hand, the standard sound pattern database contains at least standard sounds corresponding to the status of each of the body organs according to body portions to be examined, while being classified according to organs, ages, and sexes. The standard sound pattern database will be described in detail referring to an output screen described below.

The auscultatory sound database stores auscultatory sound signals input from the stethoscope, whereby the user can use the sound data stored in the memory unit 210 as required. For example, when the user performs re-diagnosis, the data stored in the auscultatory sound database allows the user to compare the present health condition with the past heath condition of a diagnosis subject, achieving more rapid and accurate diagnosis.

The information selection unit 220 is used for the user to input a manipulation signal for selecting one of standard sound patterns stored in the memory unit 210. That is, the manipulation signal is input using the information selection unit 220, for example, a well-known pointing device such as a track ball or a cursor key, and the manipulation signal is used to gain access to the standard sound patterns stored in the memory unit 210.

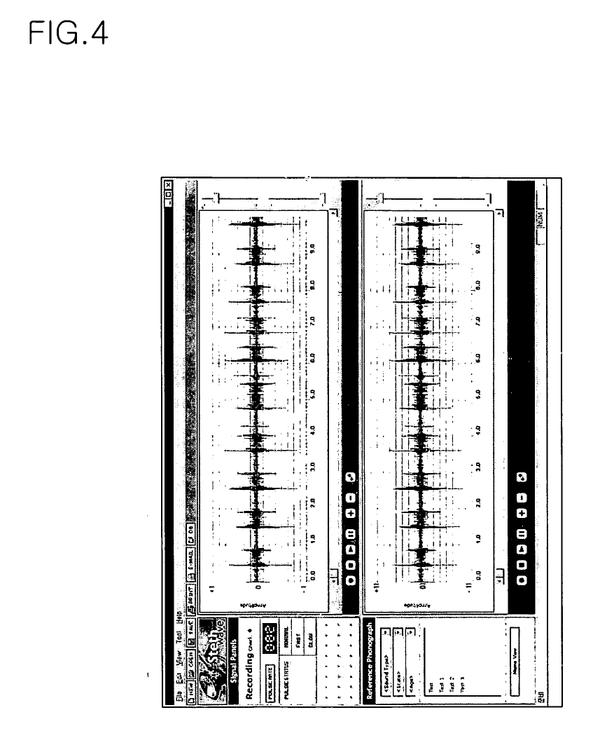

The display unit 230 displays a waveform corresponding to the auscultatory sound signal input from the stethoscope. In detail, as shown in FIG. 4a, the display unit 230 displays a standard sound pattern selected by the information selection unit, together with the sound input pattern from the stethoscope, at the same period and amplitude-scale for easy comparison therebetween, thereby allowing the user to easily judge the patient's health condition.

According to another aspect of the present invention, the display unit 230 displays the patient's pulse rate on one side of the auscultatory sound pattern screen. As a result, a number of diagnostic data are obtained through only one diagnosis to shorten the diagnosis time, thereby allowing the user to provide a higher quality of medical service to the patient.

The display unit 230 may further include a commentary display portion (not shown) for displaying a commentary about the standard sound patterns. The memory 230 may store data (i.e. explanation data) of the commentary about the standard sound patterns. Accordingly, referring to the displayed commentary, the user can explain additional diagnostic information to the patient, increasing the diagnostic accuracy.

According to another aspect of the present invention, the computer 200 further includes a communication portion 240 for transmitting a computer file containing the sound input pattern from the stethoscope, together with the diagnosis subject's identification information, to an assigned address. This allows a remote diagnosis or health consultation at home through the Internet, etc., without visiting a medical center. The communication portion may include a local area network (LAN) and a modem for data transfer.

Such a feature of the present invention is not only applied to a remote diagnosis at home, but, in the case where a patient is conveyed by an emergency vehicle such as an ambulance, the patient's auscultatory sound pattern can also be transmitted to a medical center through a wireless communication to receive a doctor's instruction, thereby achieving more efficient conveyance of patients.

FIGS. 4a and 4b are views showing screens displayed by the output system according to one embodiment of the present invention. As shown in FIG. 4a, the output system for displaying the auscultatory sound signal displays a screen of displaying an auscultatory sound pattern of the input auscultatory sound signal and its corresponding standard sound pattern. The standard sound pattern is selected by the user's manipulation of cursor movement keys according to the condition of the patient, for example, the sex, the age, and the body portion to be examined. In response to the selection, the standard sound pattern is displayed on the lower portion of the screen. Information of standard sound patterns are selected, while being classified according to sound types, for example, a heart sound generated from the heart or lung, a respiratory sound, and a mitral stenosis sound. The information is also classified according to the sound states, i.e., normal and abnormal states. In addition, the information is classified according to the patient's ages (minor and adult), and further classified according to body portions to be examined, allowing the user to select more accurately information corresponding to the condition of the patient.

On the other hand, the waveform of the auscultatory sound signal input from the stethoscope is displayed on the upper portion of the screen, and the pulse rate is output through a signal panel window of the input signal. Each lower end of the upper and lower portions of the screen includes recording, reproducing, stop, temporary stop buttons, and a section selection button, and also buttons for enlarging and reducing the pattern waveform. In addition, the noise and amplitude of a waveform in each of the upper and lower portions is adjustable, thereby achieving an easy analysis of the waveform.

In such a manner, the auscultatory sound signal indicating the patient's condition and the corresponding standard sound pattern are displayed to be compared with each other, so that the user can perform more accurate diagnosis based on the comparison therebetween, and explain or provide more helpful diagnosis information to the patient.

When a database button positioned at the top of the screen is clicked, as shown in FIG. 4b, a patient management screen is displayed, being divided into upper and lower portions. The upper portion displays the waveform of the auscultatory sound signal input from the stethoscope. The lower portion displays a patient-information input window for managing the patients to be examined. The patient-information input window is classified into sections for the user to input patient-related data such as name, sex, social security number, address, e-mail address, mobile phone number, patron's name and phone number, diagnosis queue number, and patient ID.

The modification, deletion, storing, and search of each of the input data can be performed according to the user's manipulation. A search program is provided to perform the search operation, i.e. for retrieving the records of a patient, where the patient's personal information such as ID, name, and diagnosis date can be used as a search word. The queue number of the patient can be used as a search word, but the social security number is more useful as a search word for faster and exact search.

Using a database of diagnosed patients, established in such a manner, the user can easily judge the changing condition of the patients, and also the patients can confirm their condition changes, which contributes to an increase of confidence between the user and the patients. In addition, the stored data can be provided as evidence in the case where a medical accident occurs.

Now, a detailed description will be given of a stethoscope that is applicable to an output system for displaying an auscultatory sound signal according to another embodiment of the present invention. The following description will avoid duplication with the foregoing description.

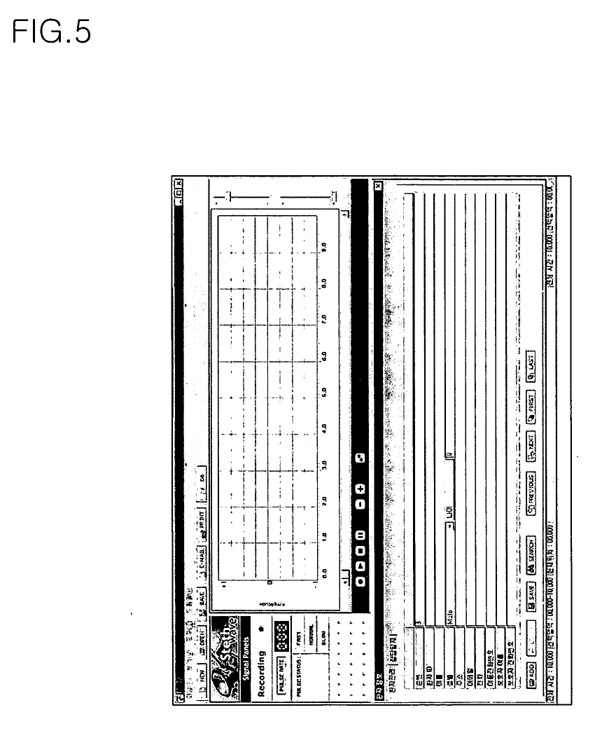

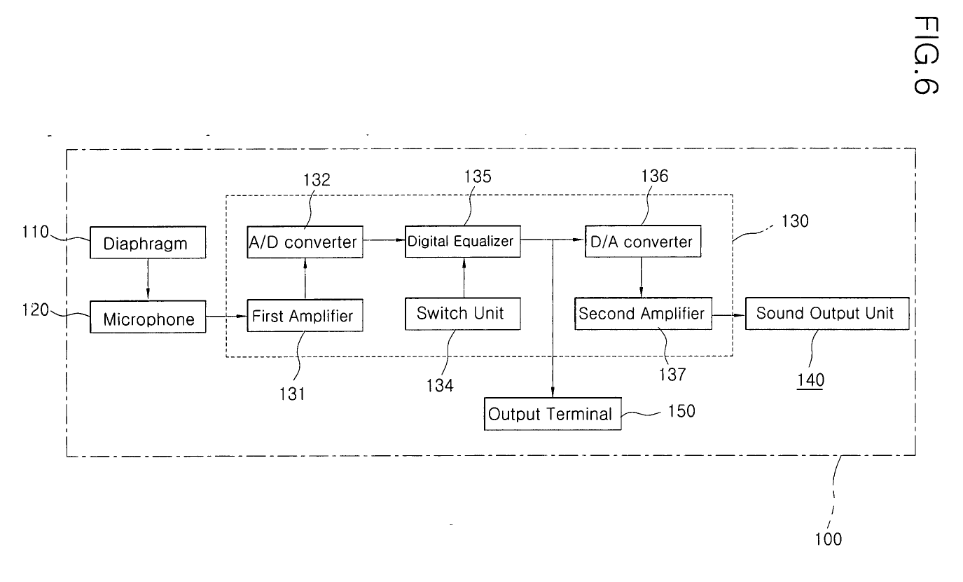

FIG. 5 is a view schematically showing the configuration of the stethoscope according to said another embodiment of the present invention. As shown in this figure, a signal process unit 130 of the stethoscope includes a first amplifier 131, an A/D converter 132, a digital equalizer 135, a switch unit 134, a D/A converter 136, and a second amplifier 137.

The A/D converter 132 converts an electrical sound signal of analog type input through the first amplifier 131 to a digital sound data, and outputs it. The digital equalizer 135 functions to separate the digital data according to frequencies, like the filter unit shown in FIG. 2. Technologies related to the digital-data filtering function of the digital equalizer 135 are well known in the art, and thus their descriptions are omitted herein.

The user can select a specific data from the digital sound signal according to the user's manipulation signal (i.e., a signal generated by the user's manipulation of a selective-mode switch). The selected data is output to a signal output unit 150 and the D/A converter 136 from the digital equalizer 135. A data converter (not shown) processes the data output to the signal output unit 150 to produce and output data capable of communicating with a computer.

The D/A converter 136 converts the digital data output from the digital equalizer 135, which corresponds to the auscultatory sound data obtained through the stethoscope 135, to an analog signal to be audible to humans. The converted electrical sound signal is amplified again with the second amplifier 137. The sound output unit converts the amplified signal to an auscultatory sound signal audible to humans, and outputs it. The computer outputs a waveform representing the digital data mentioned above.

Accordingly, an output system for displaying an auscultatory sound signal according to the present invention is advantageous in that, no matter what kind of stethoscope the system uses, the auscultatory signal is displayed together with a standard sound pattern, thereby facilitating the comparison therebetween.

Industrial Applicability

As apparent from the above description, an output system for displaying an auscultatory sound signal according to the present invention has an advantage in that, as well as the auscultatory sound is output, the auscultatory sound's pattern and its corresponding pulse rate are displayed through the monitor of a computer, thereby allowing more objective diagnosis and efficient management of patients.

In addition, because an auscultatory sound pattern and a standard sound pattern according to an auscultatory status are displayed simultaneously in such a manner that both patterns can be compared with each other, a user can perform the diagnosis of the auscultatory sound more easily and accurately.

Further, for the same reason, also the patient can confirm the present health condition of the patient with the naked eye.

Furthermore, the auscultatory sound data collected through the stethoscope can be stored in a computer or small-sized acoustic equipment to be used as required, and also can be used for a remote diagnosis through the Internet.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. An output system for displaying an auscultatory sound signal comprising:
a stethoscope including at least a) a diaphragm to be brought into contact with a specific portion of a human body so as to vibrate according to a sound generated from the body, b) a microphone for converting the vibration of the diaphragm to an electrical sound signal, c) a signal process unit for adjusting an auscultatory frequency band of the electrical sound signal from the microphone and outputting an auscultatory sound signal, d) a sound output unit for outputting a sound input from the signal process unit, and e) a signal output unit for transmitting the auscultatory sound signal output from the signal process unit to the outside; and
a computer including a) a memory unit for storing a database of standard sound patterns while being classified according to their categories, b) an information selection unit for selecting a standard sound pattern of the standard sound patterns stored in the memory unit and c) a display unit for displaying the sound input pattern from the stethoscope and the standard sound pattern selected by the information selection unit,
wherein the memory unit stores at least standard sound patterns corresponding to the status of each of the body organs according to body portions to be examined, while being classified according to organs, ages, and sexes.

2. (canceled)

3. (canceled)

4. The output system as set forth in claim 1, wherein the memory unit further includes an auscultatory sound database for storing at least auscultatory sound signals input from the stethoscope according to a manipulation signal by the user.

5. The output system as set forth in claim 1, wherein the display unit displays the standard sound pattern selected by the information selection unit and the sound input pattern from the stethoscope, at the same period and amplitude-scale.

6. The output system as set forth in claim 5, wherein the display unit outputs a pulse rate corresponding to the sound input pattern from the stethoscope.

7. The output system as set forth in claim 5, wherein the display unit further includes a commentary display portion for displaying a commentary corresponding to the standard sound pattern selected by the information selection unit, and the memory unit stores commentary data output through the commentary display portion.

8. The output system as set forth in claim 1, wherein the computer further includes a communication portion for transmitting a file containing the sound input pattern from the stethoscope, together with identification information of a diagnosis subject, to an assigned address.

9. The output system as set forth in claim 4, wherein the computer further includes a communication portion for transmitting a file containing the sound input pattern from the stethoscope, together with identification information of a diagnosis subject, to an assigned address.

10. The output system as set forth in claim 7, wherein the computer further includes a communication portion for transmitting a file containing the sound input pattern from the stethoscope, together with identification information of a diagnosis subject, to an assigned address.

11. The output system as set forth in claim 1, wherein the sound output unit includes a speaker for outputting the sound input from the sound processor, a sound collection portion for collecting sounds output from the speaker, and an earpiece having a tube for transmitting a sound output from the sound collection portion.

12. The output system as set forth in claim 1 wherein the signal process unit includes:
a first amplifier unit for amplifying the electrical sound signal generated through the microphone;
a filter unit for separating the amplified electric sound signal according to frequencies;
a switch unit for selecting an electric sound signal in a desired frequency range from the electric sound signals separated by the filter unit; and
a second amplifier for amplifying the selected electric sound signal.

13. The output system as set forth in claim 1, wherein the signal output unit includes:
an input terminal for receiving the auscultatory sound signal output from the signal process unit; and
an output terminal for outputting the auscultatory sound signal input through the input terminal to a sound card of the computer.

14. The output system as set forth in claim 13, wherein the signal output unit further includes:
a recording-instruction portion for outputting a control signal allowing the computer to record the auscultatory sound signal input from the signal process unit; and
a control signal output terminal for outputting the control signal from the recording-instruction portion to an input port of the computer.

15. The output system as set forth in claim 14, wherein the signal output unit further includes:
an AD (analog-to-digital) converter for converting the auscultatory sound signal output from the signal process unit to a digital signal and outputting the converted digital signal; and
a data converter for converting the digital signal output from the AD converter to data having a signal level and a transmission format necessary for communication with the computer, and outputting the converted data.

16. The output system as set forth in claim 1, wherein the signal process unit includes:
a first amplifier for amplifying the electrical sound signal from the microphone;
an AD converter for converting the electrical sound signal amplified by the first amplifier to a digital signal;
a digital equalizer for separating the converted digital signal according to frequencies;
a switch unit for selecting a specific digital signal in a desired frequency range from the separated digital signals;
a digital-to-analog converter for outputting the specific digital signal to the signal output unit, and simultaneously converting the selected digital signal to an electrical sound signal of analog type, according to a selection signal from the switch unit; and
a second amplifier for amplifying the converted analog sound signal.

17. The output system as set forth in claim 16, wherein the signal output unit converts the digital signal output from the signal process unit to data having a signal level and a transmission format necessary for communication with the computer, and outputting the converted data.

18. The output system as set forth in claim 17, wherein the signal output unit further includes:
a recording-instruction portion for outputting a control signal allowing the computer to record the digital signal input from the signal process unit.

19. The output system as set forth in claim 6, wherein the display unit further includes a commentary display portion for displaying a commentary corresponding to the standard sound pattern selected by the information selection unit, and the memory unit stores commentary data output through the commentary display portion.

20. The output system as set forth in claim 5, wherein the computer further includes a communication portion for transmitting a file containing the sound input pattern from the stethoscope, together with identification information of a diagnosis subject, to an assigned address.

21. The output system as set forth in claim 6, wherein the computer further includes a communication portion for transmitting a file containing the sound input pattern from the stethoscope, together with identification information of a diagnosis subject, to an assigned address.

22. The output system as set forth in claim 5, wherein the sound output unit includes a speaker for outputting the sound input from the sound processor, a sound collection portion for collecting sounds output from the speaker, and an earpiece having a tube for transmitting a sound output from the sound collection portion.

23. The output system as set forth in claim 6, wherein the sound output unit includes a speaker for outputting the sound input from the sound processor, a sound collection portion for collecting sounds output from the speaker, and an earpiece having a tube for transmitting a sound output from the sound collection portion.

24. The output system as set forth in claim 5, wherein the signal process unit includes:
a first amplifier unit for amplifying the electrical sound signal generated through the microphone;
a filter unit for separating the amplified electric sound signal according to frequencies;
a switch unit for selecting an electric sound signal in a desired frequency range from the electric sound signals separated by the filter unit; and
a second amplifier for amplifying the selected electric sound signal.

25. The output system as set forth in claim 6, wherein the signal process unit includes:
a first amplifier unit for amplifying the electrical sound signal generated through the microphone;
a filter unit for separating the amplified electric sound signal according to frequencies;
a switch unit for selecting an electric sound signal in a desired frequency range from the electric sound signals separated by the filter unit; and
a second amplifier for amplifying the selected electric sound signal.

26. The output system as set forth in claim 5, wherein the signal output unit includes:
an input terminal for receiving the auscultatory sound signal output from the signal process unit; and
an output terminal for outputting the auscultatory sound signal input through the input terminal to a sound card of the computer.

27. The output system as set forth in claim 6, wherein the signal output unit includes:
an input terminal for receiving the auscultatory sound signal output from the signal process unit; and
an output terminal for outputting the auscultatory sound signal input through the input terminal to a sound card of the computer.

28. The output system as set forth in claim 5, wherein the signal process unit includes:
a first amplifier for amplifying the electrical sound signal from the microphone;
an AD converter for converting the electrical sound signal amplified by the first amplifier to a digital signal;
a digital equalizer for separating the converted digital signal according to frequencies;
a switch unit for selecting a specific digital signal in a desired frequency range from the separated digital signals;
a digital-to-analog converter for outputting the specific digital signal to the signal output unit, and simultaneously converting the selected digital signal to an electrical sound signal of analog type, according to a selection signal from the switch unit; and
a second amplifier for amplifying the converted analog sound signal.

29. The output system as set forth in claim 6, wherein the signal process unit includes:
a first amplifier for amplifying the electrical sound signal from the microphone;
an AD converter for converting the electrical sound signal amplified by the first amplifier to a digital signal;
a digital equalizer for separating the converted digital signal according to frequencies;
a switch unit for selecting a specific digital signal in a desired frequency range from the separated digital signals;
a digital-to-analog converter for outputting the specific digital signal to the signal output unit, and simultaneously converting the selected digital signal to an electrical sound signal of analog type, according to a selection signal from the switch unit; and
a second amplifier for amplifying the converted analog sound signal.
